(19) United States
(12) Patent Application Publication
(10) Pub. No.: US 2023/0301546 A1
(43) Pub. Date: Sep. 28, 2023

(54) IMAGING HUMAN RESPIRATORY GAS PATTERNS TO DETERMINE VOLUME, RATE AND CARBON DIOXIDE CONCENTRATION

(71) Applicant: Arizona Board of Regents on Behalf of the University of Arizona, Tucson, AZ (US)

(72) Inventors: Esther M. Sternberg, Tucson, AZ (US); Jacob N. Hyde, Tucson, AZ (US); Mohammad S. Majdi, Tucson, AZ (US); Jeffrey J. Rodriguez, Tucson, AZ (US); Young-Jun Son, Tucson, AZ (US); Yijie Chen, Tucson, AZ (US)

(21) Appl. No.: 18/020,763

(22) PCT Filed: Aug. 12, 2021
(86) PCT No.: PCT/US2021/071168

§ 371 Date: Feb. 10, 2023

Related U.S. Application Data
(60) Provisional application No. 63/064,646, filed on Aug. 12, 2020.

(51) Int. Cl. A61B 5/083; A61B 5/01; A61B 5/00

(52) U.S. Cl. CPC A61B 5/0836; A61B 5/01; A61B 5/0035; A61B 5/0077; A61B 5/725; A61B 2576/00

(57) ABSTRACT

Various examples are provided related to detection and measurement of respiratory parameters. In one example, a system for monitoring an individual's respiration includes a thermal sensor that can detect CO₂, an optical sensor, and image processing circuitry that can determine a parameter of CO₂ gas being exhaled about a mouth of the individual using a thermal image captured by the thermal sensor and optical image(s) captured by the optical sensor. In other examples, a system includes an arrangement of thermal sensors and/or optical sensors that can monitor gas being exhaled about a mouth of the individual. In another example, a system includes an arrangement of thermal and optical sensors and image processing circuitry that can determine, e.g., a flow field and/or distribution of gas being exhaled about a mouth of the individual based upon thermal images and optical images captured by the arrangement of thermal and optical sensors.

### CROSS REFERENCE TO RELATED APPLICATIONS

This application claims priority to, and the benefit of, co-pending U.S. provisional application entitled “Imaging Human Respiratory Gas Patterns to Determine Volume, Rate and Carbon Dioxide Concentration” having Ser. No. 63/064,646, filed Aug. 12, 2020, which is hereby incorporated by reference in its entirety.

### BACKGROUND

Acute respiratory distress syndrome (ARDS) is a major complication in 17-29% of patients with severe COVID-19 pulmonary disease, which can manifest shortly after the onset of difficulty breathing. In 138 patients hospitalized in Wuhan for pneumonia, difficulty breathing developed after a median of five days from symptom onset and patients were admitted to hospital after a median of seven days of symptoms. A subsequent retrospective study of 191 adult patients hospitalized with COVID-19 disease reported a median time from the onset of symptoms to the development of ARDS of 12 days with a range of 8 to 15 days. Among 393 patients in New York City, 79.4% presented with cough and 56.5% with dyspnea. Among these, 130 patients required invasive mechanical ventilation due to respiratory failure.

According to the Berlin definition, ARDS is characterized by the following criteria: progressive respiratory symptoms within one-week, bilateral opacities on imaging, and edema not fully explained by cardiac failure or fluid overload. COVID-19 related ARDS is different from ARDS caused by other diseases, and can present with relatively mild symptoms, mainly fever and a dry cough without overt respiratory distress. Dyspnea, myalgia, weakness, and/or chest tightness are each experienced by approximately ⅓ of all patients. Challenging the monitoring of COVID-19 patients is that even those with initial mild presentations may decompensate quickly. Thus, all symptomatic patients should have close monitoring, especially for blood carbon dioxide levels to determine the need for mechanical ventilation.

In progressive COVID-19 related ARDS, patients experience dyspnea, increased respiratory rate and respiratory distress. The distinguishing characteristics of COVID-19 pneumonia are significant hypoxemia with close to normal respiratory compliance. CT imaging typically reveals bilateral ground glass opacities. As ARDS onset can occur very quickly, with a median time of 8 days after the appearance of mild respiratory symptoms of COVID-19, it is important to have ways to monitor and quickly identify respiratory decline before it reaches a critical level.

Current evaluation of respiratory distress and failure utilizes cumbersome and relatively invasive pulmonary function tests such as, e.g., spirometry, lung volume, and lung diffusion capacity. However, patients with severe ARDS may be unconscious or too weak to effectively perform these tests, and likely cannot be transported to testing equipment. Furthermore, administering these tests requires close contact of healthcare personnel with patients, which increases the risk for healthcare personnel contracting viral infection.

### SUMMARY

Aspects of the present disclosure are related to detection and measurement of respiratory parameters. The disclosed non-invasive, remote sensing, multi-sensor respiratory imaging system can be used for continual measurement of respiratory parameters of exhaled respiratory gases without contact Using the disclosed technology, changes in respiratory patterns and CO₂ concentration in exhaled respiratory gases can be continually monitored, allowing for early warnings that can alert healthcare personnel to the beginnings of respiratory failure. Such early detection can allow therapeutic interventions to be implemented before respiratory failure has progressed, thus reducing the morbidity, mortality, and severity of acute respiratory distress syndrome (ARDS) secondary to corona viruses, such as COVID-19. Furthermore, by employing non-invasive, imaging systems, protection is provided to healthcare providers by reducing close contact with infected patients that is currently needed to monitor their respiratory status. This can reduce the chance of healthcare personnel themselves becoming infected with a virus such as SARS-CoV-2, and potentially themselves developing ARDS.

In one aspect, among others, a system for monitoring an individual's respiration comprises a thermal sensor configured to detect CO₂ gas; at least one optical sensor; and image processing circuitry configured to determine a parameter of CO₂ gas being exhaled about a mouth of the individual based upon a thermal image captured by the thermal sensor and at least one optical image captured by the at least one optical sensor. In one or more aspects, the system can comprise processing circuitry configured to provide an indication of onset of acute respiratory distress syndrome (ARDS) in the individual based upon the parameter. The image processing circuitry can be configured to provide an indication of onset of acute respiratory distress syndrome (ARDS) in the individual based upon the parameter. The thermal sensor can comprise a camera positioned about the individual, and the at least one optical sensor can comprise at least one optical camera positioned about the individual. The thermal camera can comprise a filter to detect the exhaled CO₂ gas. The filter can be a bandpass filter with a center wavelength between about 4.1 μm and about 4.5 μm.

In various aspects, the parameter can be determined by a background-oriented schlieren (BOS) technique. The parameter can be determined by combining information determined from the thermal image and information determined from the at least one optical image. The resolution of the at least one optical image can be at least four times the resolution of the thermal image. The image processing circuitry can determine a point cloud representation of a surface shape of the exhaled CO₂ gas. The system can comprise early warning processing circuitry configured to provide an early warning indication in response to the exhaled CO₂ gas reaching or exceeding a predefined level of CO₂ concentration, CO₂ volume, or rate of change of the CO₂ concentration or CO₂ volume. The image processing circuitry can be configured to provide an early warning indication in response to the exhaled CO₂ gas reaching or exceeding a predefined level of CO₂ concentration, CO₂ volume, or rate of change of the CO₂ concentration or CO₂ volume. The image processing circuitry and/or the early warning processing circuitry can be remotely located. The early warning indication can be communicated to a user device for display. The parameter can comprise volume or three-dimensional (3D) information of the exhaled CO₂ gas. The parameter can be based upon the thermal image and a plurality of optical images captured by the at least one optical sensor at a plurality of time points. The parameter can be based upon a plurality of thermal images captured by the thermal sensor at a plurality of time points and the at least one optical image. The parameter can comprise a rate of change of the exhaled CO₂ gas. The at least one optical sensor can comprise a plurality of optical sensors, and the image processing circuitry can be configured to determine the parameter of CO₂ gas being exhaled about a mouth of the individual based upon the thermal image captured by the thermal sensor and a plurality of optical images captured by the plurality of optical sensors. The parameter can be determined by a background-oriented schlieren (BOS) technique. The parameter can comprise volume or three-dimensional (3D) information of the exhaled CO₂ gas.

In another aspect, a system for monitoring an individual's respiration comprises an arrangement of thermal and optical sensors configured to monitor respiration of an individual, where the thermal sensors are filtered to detect CO₂; and image processing circuitry configured to determine a flow field or three-dimensional (3D) distribution of gas being exhaled about a mouth of the individual based upon thermal images and optical images captured by the arrangement of thermal and optical sensors. In one or more aspects, the system can identify onset of ARDS in the individual based at least in part upon the flow field or 3D distribution of gas. The arrangement of thermal and optical sensors can comprise one or more thermal cameras and one or more optical cameras positioned about the individual. Individual thermal cameras of the one or more thermal cameras can comprise a filter to detect the CO₂. The filter can be a bandpass filter, e.g., with a center wavelength between about 4.1 μm and about 4.5 μm. A property of CO₂ gas being exhaled about the mouth of the individual can be determined based upon thermal images. The optical images can be provided by the one or more optical cameras with a background-oriented schlieren (BOS) technique.

In various aspects, the flow field or 3D distribution of gas can be determined by combining information determined from the thermal images and information determined from the optical images, e.g., using the BOS technique. The resolution of the optical images (e.g., one megapixel) can be at least four times the resolution of the thermal images. A point cloud representation of a surface shape of a bubble of the exhaled gas can be generated based upon the optical images. Processing circuitry can be configured to provide an early warning indication in response to the exhaled gas reaching or exceeding a predefined level of CO₂ concentration, CO₂ volume, or rate of change of the CO₂. A remotely located computing device can comprise the processing circuitry configured to determine the flow field or 3D distribution of gas and provide the early warning indication. The early warning indication can be communicated to a user device for display.

In another aspect, a system for monitoring an individual's respiration comprises an arrangement of thermal sensors configured to monitor respiration of an individual where the thermal sensors are filtered to detect CO₂. In one or more aspects, the arrangement of thermal sensors can comprise a plurality of thermal cameras positioned about the individual. Individual thermal cameras of the plurality of thermal cameras can comprise a filter to detect CO₂. The filter can be a bandpass filter. A property of CO₂ gas being exhaled about the mouth of the individual can be determined based upon thermal images captured by the arrangement of thermal sensors.

In another aspect, a system for monitoring an individual's respiration comprises an arrangement of optical sensors configured to monitor gas flow being exhaled about a mouth of the individual. The arrangement of optical sensors can comprise a plurality of optical cameras positioned about the individual. Optical images can be captured by the plurality of optical cameras with a background-oriented schlieren (BOS) technique. The gas flow being exhaled about the mouth of the individual can be determined based upon the optical images. Resolution of the optical images can be at least one megapixel. A point cloud representation of a surface shape of a bubble of the gas flow being exhaled about the mouth of the individual can be generated based upon the optical images.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of background-oriented schlieren (BOS) imaging of convective air flow, which can also be applied to imaging human respiration, in accordance with various embodiments of the present disclosure.

FIG. 2 illustrates an example of CO₂ bubble concentrations during exhalation, in accordance with various embodiments of the present disclosure.

FIG. 3 is an image of an S8 CO₂ sensor coupled to processing circuitry for measurement of CO₂ concentrations, in accordance with various embodiments of the present disclosure.

FIGS. 4A-4C illustrate examples of CO₂ sensor placement and detected CO₂ accumulation and distribution during respiration testing, in accordance with various embodiments of the present disclosure.

FIGS. 5A and 5B illustrate examples of CO₂ sensor placement and detected CO₂ accumulation during respiration testing of an individual, in accordance with various embodiments of the present disclosure.

FIG. 6 illustrates an example of thermal imaging for detection of exhaled gas, in accordance with various embodiments of the present disclosure.

FIGS. 7A and 7B illustrate an example of an early warning system and outputs, in accordance with various embodiments of the present disclosure.

FIG. 8 illustrates an example of a CO₂ sensor and thermal camera in several possible positions for calibration and training of the respiratory imaging system, in accordance with various embodiments of the present disclosure.

### DETAILED DESCRIPTION

Disclosed herein are various examples related to imaging exhaled human respiratory gases to measure respiratory parameters such as, e.g., volume, respiration rate, carbon dioxide (CO₂) concentration, and combinations thereof. For example, a multi-sensor respiratory imaging system, utilizing thermal sensors and optical imaging sensors, can be used to capture images of exhaled human respiratory gases through two complementary imaging techniques: thermal imaging using, e.g., one or more thermal cameras with CO₂-specific filtering for imaging the CO₂ gas component and optical imaging using, e.g., one or more optical cameras configured for imaging the whole exhaled gas bubble using, e.g., a background-oriented schlieren (BOS) technique. Thermal imaging sensors include cameras or other sensors configured for infrared thermography or other infrared imaging techniques. Optical imaging sensors include cameras or other sensors configured for imaging in the visible light spectrum. The system includes image processing circuitry to process the captured images to compute three-dimensional (3D) models of gas (e.g., structure, shape, concentration, and motion) and compute respiratory parameters such as, e.g., volume, rate, CO₂ concentration, and combinations thereof. The system can include an output device (e.g., computer display or monitor) for visualizing the captured images and the computed 3D models and parameters of gas flow and for early warning signaling when respiratory parameters and exhaled CO₂ reach dangerous levels. Reference will now be made in detail to the description of the embodiments as illustrated in the drawings, wherein like reference numbers indicate like parts throughout the several views.

Current evaluation of respiratory distress and failure utilizes MRI, X-ray or CT imaging, blood tests and cumbersome and relatively invasive pulmonary function tests—e.g., blood gases, spirometry, lung volume, and lung diffusion capacity. There are no universally defined monitoring protocols to guide clinical management. The National Health Commission of China primarily bases treatment on three categories of patient oxygenation index [Partial pressure of O₂ (PaO₂)/Fraction of inspired oxygen (FiO₂) (P/F ratio)] on positive end expiratory pressure (PEEP). ARDS can be categorized as mild, moderate, or severe by measuring lowered P/F ratios. Generally, routine blood tests and chest imaging are also used to monitor progression in infected patients.

Exhaled breath analysis may be used to diagnose ARDS and other lung diseases. While most prior devices measure exhaled volatile organic compounds, measurement of exhaled CO₂ can an effective method to measure lung function. However, the devices for collection of exhaled respiratory gases are invasive or cumbersome, some being applicable only in intubated patients, and others requiring mouth devices for gas collection. Methods for measuring respiratory rate include a nasal cannula and a thermistor that measures the temperature change between the inhaled air and exhaled respiratory gases. Most clinical approaches for diagnosis of ARDS require patients' active participation to achieve diagnostic test accuracy. Lung imaging typically needs transport to radiology which exposes healthcare providers to possible viral exposure. Moreover, transport for imaging as well as invasive testing in patients with severe ARDS increases the risk of secondary bacterial infection to these patients, a known contributor to mortality.

In this disclosure, a remote detection technology using a multi-sensor respiratory imaging system for continual measurement of respiratory parameters of human exhaled respiratory gases is described. The multi-sensor respiratory imaging system allows for monitoring without patient contact.

Thermal Imaging of Human Respiration: Thermal imaging using, e.g., one or more FLIR® infrared cameras can be used to obtain a 2D projection of a heat source. Thermal imaging is useful for detection of human respiration based on temperature patterns, given its ability to monitor respiration unobtrusively and non-invasively. Computer algorithms can be utilized to analyze such data. However, such imaging and algorithms do not detect CO₂ specifically. To accomplish this, a CO₂ filter (e.g., a bandpass filter, e.g., with a center wavelength between about 4.1 μm and about 4.5 μm) can be added to the FLIR camera to isolate CO₂ gas. During human respiration, CO₂ can build up around the face of a person, creating a “CO₂ bubble”. Thermal imaging with isolated CO₂ filtration can be used to visualize and measure CO₂ production, accumulation, and dissipation. The thermal imaging with a CO₂ filter offers a non-invasive and remote way to monitor CO₂ production, accumulation, and dissipation.

Thermal imaging captures infrared images, which can be used for detection of human respiration based on temperature patterns, providing the ability to monitor respiration unobtrusively and non-invasively. Thermal imaging cameras (e.g., FLIR cameras) convert light in the infrared (IR) region of the electromagnetic spectrum to a visual image that depicts thermal variations in the field of view. IR radiation is emitted by all objects with a temperature above absolute zero, and radiation amounts increase with temperature. However, these infrared thermal imaging cameras can only provide a two-dimensional (2D) projection of the heat source and do not directly measure the 3D geometry of the exhaled CO₂ bubble. Multi-view thermal imaging can be employed to derive a 3D model of a CO₂ gas bubble. Using images from two or more thermal cameras, photogrammetry (e.g., stereoscopy) can be used to reconstruct the 3D geometry of an imaged object such as a CO₂ gas bubble.

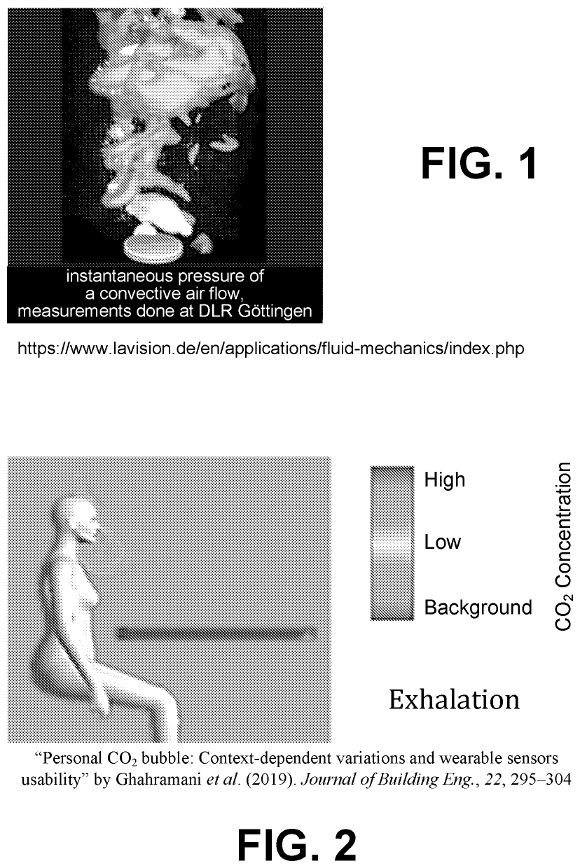

BOS Imaging of Human Respiration: An imaging system that comprises optical imaging using one or more optical cameras along with the BOS technique has proven to be a fast and easy technique for visualization of transparent media such as gas, which makes it ideal for imaging gas emissions in human respiration. BOS imaging utilizes data from a digital camera to visualize the fluid-dynamic characteristics of transparent media such as gas. The BOS method detects the refractive disturbances caused when imaging a background (e.g., comprising non-uniform features such as random speckle patterns) in the presence of a foreground object of interest such as a gas bubble. A reference image known as a tare image is taken prior to introducing the foreground object, and a data image is captured in the presence of the foreground object. Image processing techniques are then used to compare the tare image and the data image to determine a reconstructed image that contains information about the foreground object, such as a flow field that indicates the speed and direction of flow at various points across the gas bubble. Due to the importance of volumetric measurement techniques in capturing the 3D structure of the gas flow, a 3D extension of this method (e.g., the 3D BOS method using at least two optical cameras) can be used to derive a 3D geometrical model to improve the accuracy in quantitative measurements. Using known tomographic reconstruction techniques, 3D BOS imaging measures the fluid-dynamic characteristics in 3D using images from BOS projections from two or more cameras. FIG. 1 illustrates an example of BOS imaging of convective air flow, which can also be applied to imaging human respiration. By utilizing this technique along with a thermal imaging system, the combination can be used to obtain a high-resolution 3D visualization of an exhaled CO₂ bubble and thus accurately detect any signs of respiratory decline. For example, the LaVision DaVis software with BOS module may be used.

3D Mapping of Surface Temperature: Three-dimensional (3D) mapping of surface temperature distribution can be utilized for imaging exhaled human respiratory gases to measure volume, rate, CO₂ concentration, and combinations thereof. For example, 3D-BOS imaging can be used in conjunction with thermal imaging to acquire 3D surface information, yielding 3D models including geometry and temperature. Alternatively, photogrammetry (e.g., stereoscopy) of multi-view thermal images or range imaging (e.g., structured light imaging, time-of-flight imaging, laser scanning) may be used to acquire 3D surface information, yielding 3D models. Intensity data from 2D images (e.g., thermal and/or infrared) may be combined with 3D geometrical models via texture mapping to yield a heat distribution map containing both appearance and temperature information.

CO₂ Detection and Visualization: It has been shown that during exhalation the “CO₂ bubble” around a person's face has CO₂ concentrations ranging from, e.g., 400 to 1200 ppm. FIG. 2 illustrates an example of the CO₂ bubble concentrations during exhalation. Current CO₂ detection sensors utilize infrared spectral measurement to accurately measure CO₂ levels. These sensors have varying ranges of sensitivity, some more sensitive at lower ranges equivalent to room air (e.g., 400-2000 ppm CO₂), and others more sensitive at higher ranges for use in industrial settings (e.g., >10,000 ppm CO₂). To accurately and sensitively measure the exhaled CO₂ bubble, an arrangement of sensors close to the mouth can be used. However, this is too cumbersome for clinical settings and would likely interfere with respiration. Rather, remote thermal imaging can be used to overcome this drawback. For example, a FLIR camera may be fitted with a CO₂ filter (e.g., a bandpass filter with center wavelength between about 4.1 μm and about 4.5 μm) to specifically and remotely image CO₂. Thermal imaging with isolated CO₂ filtration, in conjunction with optical imaging and image processing circuitry, can generate a 3D model of CO₂. Isolating CO₂ from other respiratory gases with an appropriate CO₂ filter can be used to continually monitor respiratory status remotely and non-invasively.

Combination of Thermal and Optical Information: Even in the presence of recent technological advancement in infrared cameras, they still lack the resolution (i.e., number of pixels) that can be obtained in conventional RGB cameras (e.g., 16-20 megapixels). For example, the currently available models of FLIR cameras include resolutions ranging from 80×60 pixels (0.005 megapixel) up to 1024×768 pixels (0.8 megapixel). Combining the CO₂ information extracted from the thermal imaging system with a 3D model of exhaled respiratory gases derived from the optical imaging system (e.g., a BOS imaging system) by fusing the relevant CO₂ information onto the 3D model, can yield an extensive amount of valuable information that may dramatically improve the visualization of human CO₂ respiration and thus provide a far more accurate prediction of CO₂ respiratory decline.

Image Processing Circuitry: The 2D images captured by the optical imaging system and the thermal imaging system, as well as a reconstructed 3D model, can be processed by the image processing circuitry (e.g., one or more processors or application-specific integrated circuits or graphics processing units (CPUs) or CPUs or combinations thereof, such as processing circuitry in a smartphone, tablet, laptop, or other client computing device). The functionality of the image processing circuitry may be determined by software or hardware description language code, and may include, e.g., image denoising, image enhancement, image registration, image segmentation, and 3D reconstruction. Image denoising can be performed using, e.g., low-pass filtering. Image enhancement can be performed using, e.g., total variation optical flow. For example, the software may include FLIR ResearchIR Max and LaVision DaVis with BOS module. Image registration can be performed using, e.g., analysis of digital image correlation. Image segmentation can be performed using, e.g., Otsu's thresholding technique and connected components analysis. Multi-view depth estimation can be achieved by utilizing images acquired from optical cameras, or alternatively, from thermal cameras. The multi-view depth (distance) estimates can be used to reconstruct a dense 3D representation of a gas bubble based on camera parameters and poses. Information derived from thermal images can be overlayed onto the optical images by using ray tracing techniques by first segmenting an estimated area of gas bubble in both thermal and optical images and then performing an intensity-based, multi-modality image registration between the thermal and optical images. Thermal information can also be overlaid or mapped onto the 3D gas bubble reconstructed using images obtained from the optical imaging system by using, e.g., ray tracing techniques. An estimated volume of the exhaled gas bubble can be computed from a single optical image by first performing image segmentation to determine a projected 2D gas bubble corresponding to the 3D gas bubble, then computing an area of the projected 2D gas bubble, followed by computing an average radius of the projected 2D gas bubble, and then extrapolating to determine an estimated volume of the 3D gas bubble by using a typical shape of the 3D gas bubble (e.g., spherical). For example, if A is the computed area of the projected 2D gas bubble and if the 3D gas bubble is assumed to have a spherical shape and the projected 2D gas bubble is assumed to have a circular shape, then the average radius r of the projected 2D gas bubble is given by:

r=\sqrt{A/π},

and the estimated volume V of the 3D gas bubble is given by:

$$V = \frac{4}{3} π {r}^{3} .$$

This volume estimation procedure can be modified to include information from the thermal images in order to compute an estimated volume of the 3D CO₂ gas bubble. Alternatively, a plurality of optical sensors can be used to determine a 3D geometrical model of the 3D gas bubble and a more accurate volume estimate. Specifically, well-known 3D reconstruction techniques can be used to compute a 3D model from 2D images from a plurality of optical cameras—e.g., correspondence matching and disparity analysis. Techniques such as stereoscopic matching and spatial jitter analysis may be used to compute a 3D model of the surface of the gas bubble. This can yield a point cloud representation of the surface shape of the gas bubble.

Temporal Image Analysis: In another aspect, a system for monitoring an individual's respiration uses images from an array of thermal and optical sensors to monitor temporal parameters of respiration of an individual. The image processing circuitry may be used repeatedly at a plurality of time points to determine a gas flow field and 3D distribution of gas being exhaled about a mouth of the individual at each time point based upon thermal and optical images captured by the array of thermal and optical sensors. By determining variations in the gas flow field and 3D distribution between different time points, the image processing circuitry may compute various temporal parameters of respiration, such as, e.g., a rate of change in the gas volume, a rate of change in the respiration rate, and a rate of change in the CO₂ concentration. An algorithm such as, e.g., the pyramidal Lucas-Kanade method can be used to track the contour of exhaled respiratory gases and estimate the movement of exhaled respiratory gases across various time points.

Mini CO₂ Sensors for Calibration and Validation: Studies were carried out to identify miniature CO₂ sensors suitable for measurement of CO₂ at close range. It was found that the S8 Miniature 10,000 ppm (1%) CO₂ Sensor, which uses nondispersive infrared measurement, was the most consistently sensitive in the lower CO₂ ranges of interest compared to other available sensors in this range (e.g., K30 10,000 ppm CO₂ Sensor (GasLab), SCD-30 CO₂ and humidity sensor), and also fulfilled the data download capabilities needed to connect with other respiratory monitoring equipment.

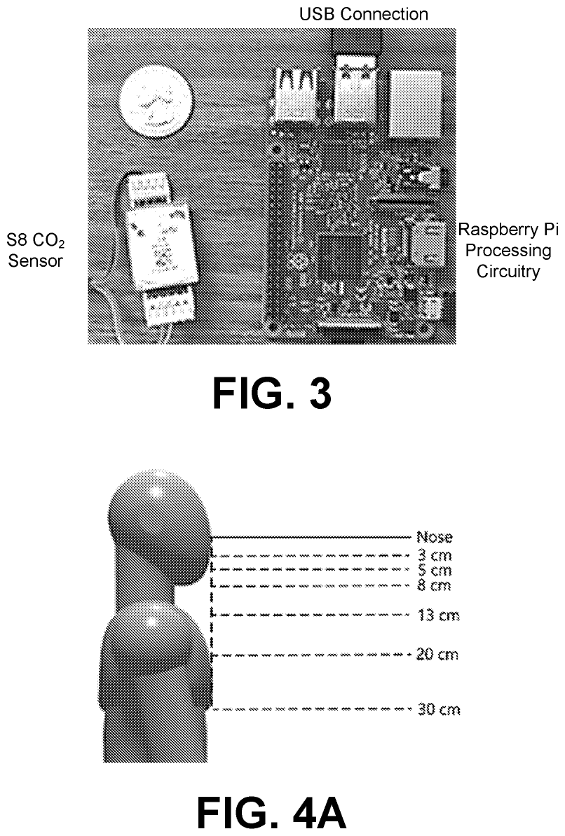

CO₂ Sensor Testing and Placement: In order to determine effective sensor placement, the S8 Miniature 10,000 ppm (1%) CO₂ sensors were used to detect CO₂ concentration in the range of 400 to 10,000 ppm. The CO₂ sensor has a small size (32.7×19.7×8 mm), high data collecting frequency (1.5 s per reading) and high data accuracy (±70 ppm±3% of reading). A sensor controller (e.g., a processor such as a Raspberry Pi) can be used to collect data inputs from the sensor through, e.g., a USB or other appropriate connection, converting readings into text or spreadsheet files to store the data. FIG. 3 is an image showing an example of an S8 CO₂ sensor coupled to a sensor controller (e.g., a processor such as a Raspberry Pi) through a USB port. The sensor controller can also allow for reception of data through a wireless connection (e.g., Bluetooth® or WLAN) remotely.

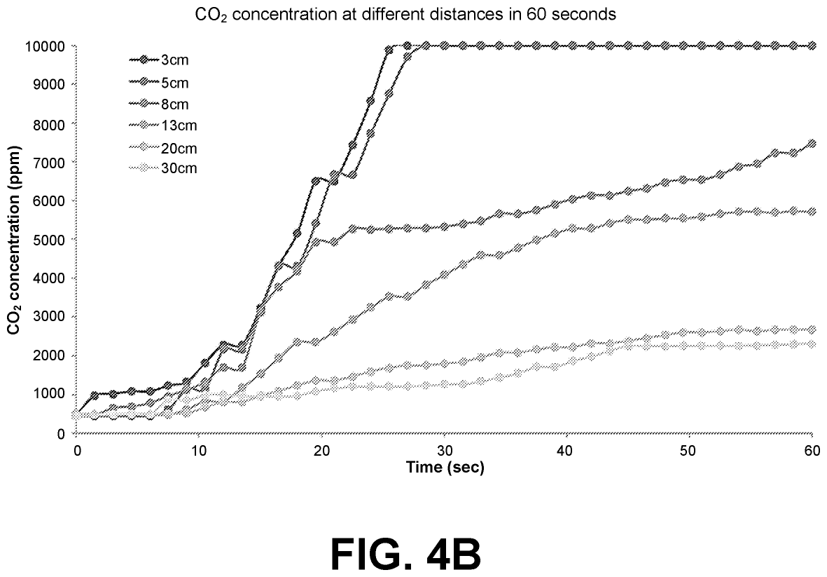

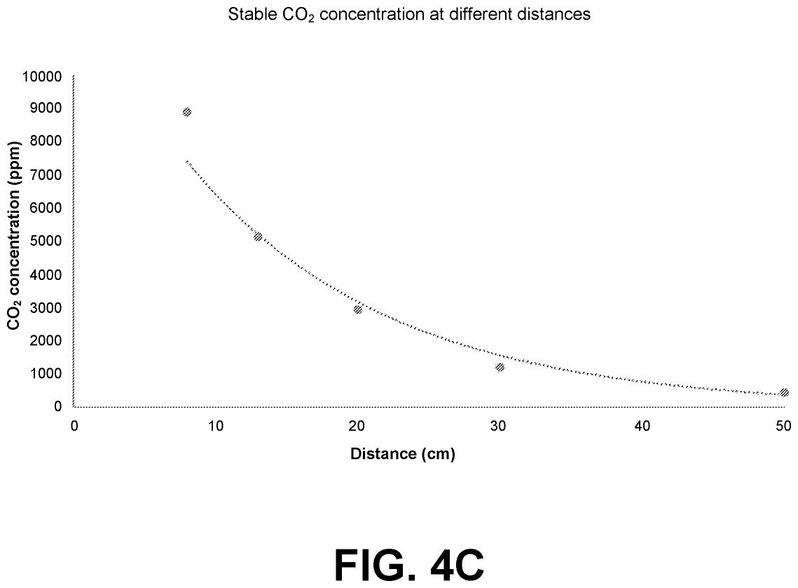

In a preliminary experiment, a participant was requested to sit in a chair and keep the upper body straight. The CO₂ sensor was placed at different distances (3, 5, 8, 13, 20, and 30 cm) below the nose with the diffusion area up. FIG. 4A illustrates the CO₂ sensor placement during the testing. At each distance, the measuring time was 5 minutes (1.5 s per reading), and before each start, the CO₂ sensor was reset in environment air (around 450 ppm). The testing results validated that the CO₂ concentration at different distances had different accumulation speeds and the CO₂ concentrations at different distances were subject to an exponential distribution rather than a linear distribution. FIGS. 4B & 4C illustrate the CO₂ accumulation over time and the exponential distribution at the various distances, respectively.

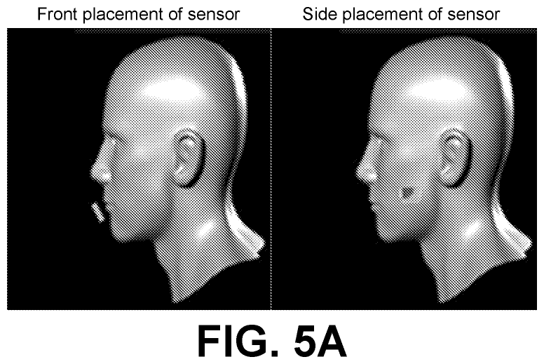

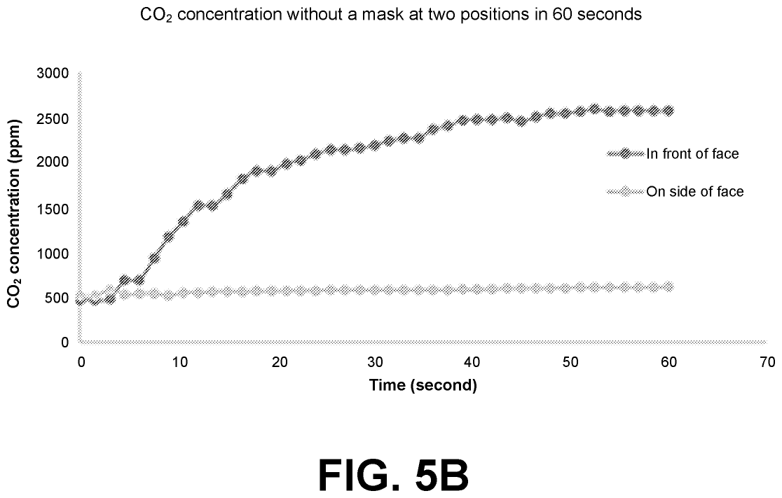

Experiments were also conducted with S8 Miniature 10,000 ppm (1%) CO₂ sensors to detect CO₂ concentrations in the range of 400 to 10,000 ppm by placing the sensors in two positions on the side of the face and in front of the mouth. FIG. 5A illustrates the sensor placement in front and on the side of the mouth (left and right, respectively). FIG. 5B shows the CO₂ accumulation results, with the CO₂ concentration in front of the mouth being higher, as would be expected.

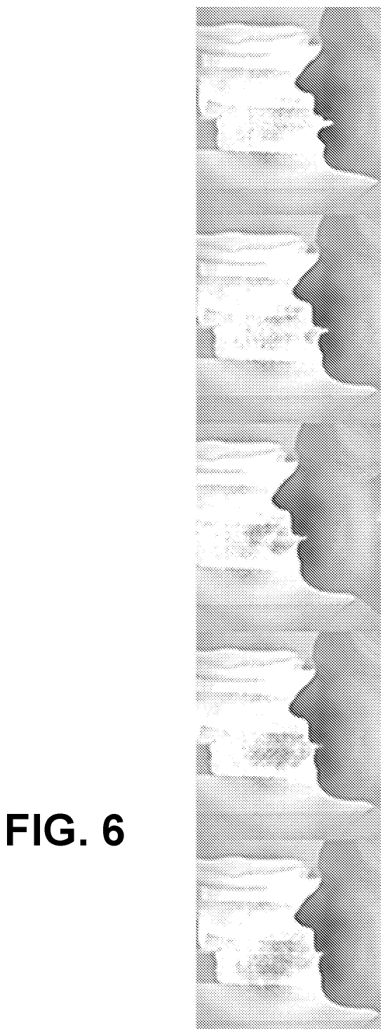

Thermal Imaging Studies: Preliminary tests were conducted in order to establish the method for utilizing the FLIR camera to visualize exhaled respiratory gases and CO₂. Thermal imaging was used to image human respiration with a FLIR A6703sc camera and a 50 mm f/4 lens in a laboratory environment. FIG. 6 shows a series of examples of thermal images of exhaled gas. A CO₂ filter (e.g., a bandpass filter with a center wavelength between about 4.1 μm and about 4.5 μm) may be affixed to the lens of the FLIR camera to capture CO₂ exhalation. In these studies, thermal image data was fed via CAT6 interface to a graphics processing unit (GPU) and recorded using FLIR ResearchIR Max software. A CPU may also be used. In other studies, an algorithm was developed in MATLAB that utilizes image analysis tools to visualize thermal changes at a micro-scale.

Room Air CO₂ and Ventilation Velocities: A Particle Image Velocity TSI camera system and other velocity sensors, as well as CO₂ sensors were utilized to measure room air and ventilation velocity and CO₂ concentrations.

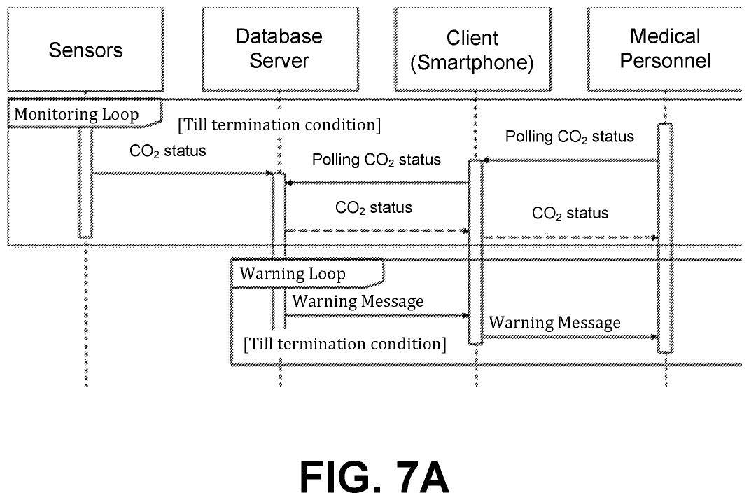

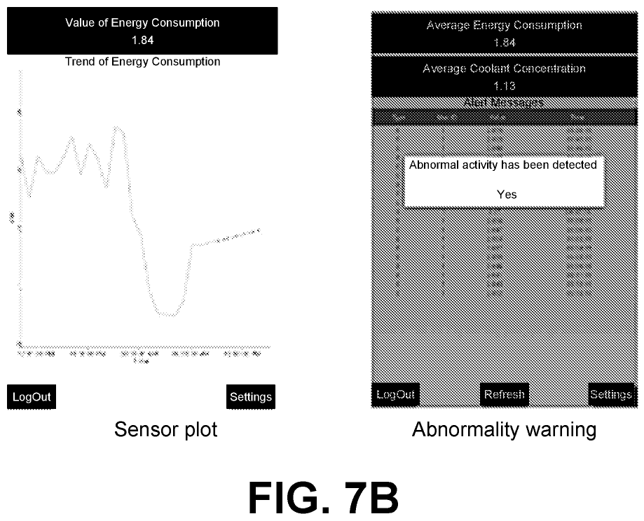

Early Warning Indication: An early warning system can be developed to provide an indication of the condition of an individual. FIGS. 7A and 7B show an example of the early warning system that outputs information (e.g., sensor plot or abnormality warning) that can be displayed to medical personnel or other user via the display device or a user device such as, e.g., a smartphone, tablet, laptop or other client computing device.

Multi-Sensor Respiratory Imaging System Design. A multi-sensor thermal and optical imaging system (e.g., using BOS imaging) can be used to accurately compute respiratory parameters such as the volume, respiratory rate and CO₂ concentration of exhaled respiratory gases, or rates of change of respiratory parameters, enabling continual monitoring of respiratory patterns and CO₂ concentration and provide early warning if one or more monitored parameters approach or enter clinically dangerous ranges. The remote, non-invasive multi-camera-based device and early warning system can continually monitor and detect early respiratory failure in patients with respiratory disease such as COVID-19. This can allow for faster application of medical countermeasures and treatment as well as monitoring of recovery in infected patients, ultimately enhancing the ability to reduce morbidity and mortality. This system may be applied to any cause of respiratory failure and can be adapted for use with readily available smartphone/tablet technology that is easily set up in hospitals/clinics around the world.

A multi-sensor respiratory imaging system comprising, e.g., FLIR thermal cameras with a CO₂ filter and optical digital cameras for use with background-oriented schlieren (BOS) technique can be arranged to image exhaled human respiratory gases and compute respiratory parameters—e.g., volume, respiratory rate, CO₂ concentration, and rate of change of the CO₂ concentration or CO₂ volume. These two complementary imaging techniques can be combined through processing to perform 3D reconstruction and gas flow calculation. The CO₂ concentration of exhaled respiratory gases can be visualized using one or more thermal cameras (e.g., FLIR cameras) fitted with CO₂ filters to isolate the CO₂ gas. A cold background may be used to improve thermal image contrast. Visualization of the 3D surface boundary of exhaled respiratory gases can be achieved using the BOS imaging system. Low-resolution thermal data containing CO₂ gas information and high-resolution data, determined from the optical images, containing information about the whole exhaled respiratory gas bubble can be combined through further processing. The data collected from the CO₂ visualization and airflow monitoring can be utilized to provide an early warning alarm that can be indicated through existing smartphone/tablet devices. Placement of the sensors and variables related to human subjects (e.g., movement, temperature differences in exhaled respiratory gases, and sex differences in exhaled gas volumes) may be monitored and accounted for by the application.

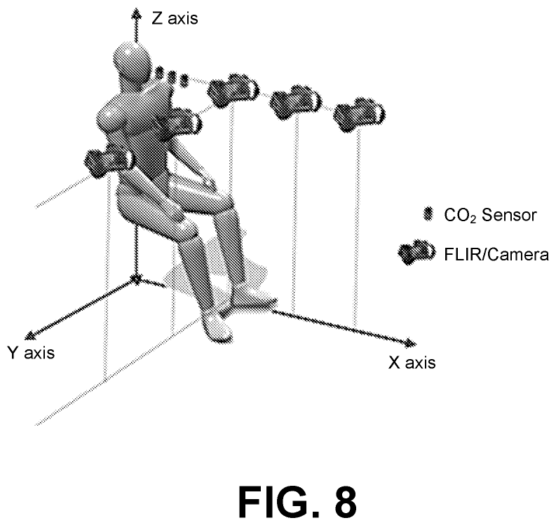

Validation and calibration of camera-derived CO₂ concentration and respiratory rate can be carried out against sensors such as, e.g., gold-standard measurement devices. For example, known concentrations of CO₂ can be used to calibrate CO₂ concentration gradients detected by the one or more FLIR cameras with CO₂ filter, and in the refinement of algorithms to measure volume and respiratory rate of total gases imaged using the imaging system (e.g., a BOS imaging system). CO₂ gas canisters containing different concentrations of CO₂ can be incorporated into a head and torso model with accurate nose and mouth apertures typically used in medical simulation resuscitation training. An array or arrangement of S8 mini-CO₂ sensors can be placed at fixed distances in front of the simulation model mouth and nose aperture in order to measure CO₂ concentrations at different distances, which can then be correlated with thermal imaging acquired through thermal cameras at different locations about the model. FIG. 8 illustrates the positioning of the CO₂ sensors and thermal cameras. The S8 sensors can be placed at varying distances to establish the range at which CO₂ concentration can be detected. In addition to fixed distances, lateral view, top view, and front view can also be positioned at different distances. Similarly, the optical imaging system can be positioned at various positions and distances.

Different amounts of CO₂ with known standard CO₂ concentrations can be expelled through the aperture, in order to develop a gradient of CO₂ concentrations that can be imaged by the imaging system. An auto-regressive integrated moving average (ARIMA) can be used to analyze the data collected from different CO₂ sensors to create a temporal model of stable CO₂ flow rate changes. The series of data from different time points within each stable breathing cycle can be collected from the CO₂ sensors and used to calibrate the CO₂ concentration reading. Multiple regression models may be developed to relate data collected from different CO₂ sensors with respect to their distance and position. Whenever the CO₂ being exhaled by a patient does not follow the expected flow rate predicted by the ARIMA model, it would mean that there has been a change from the stable healthy expiration, and may be used as an indicator to warn medical personnel.

3D reconstruction of exhaled respiratory gases can be based upon two or more optical images, and the reconstructed 3D model can be combined with information from the thermal (e.g., FLIR) cameras (which may include CO₂ filters) that can be used to isolate CO₂ gas. A 3D model of the surface of exhaled CO₂ gas can be reconstructed using this information. The multi-camera system can be positioned at different angles and distances that may be adjusted to find an optimum placement. The effect of room air flow on exhaled respiratory gases and the relationship of the ventilation in the room, including any turbulence effects on the exhaled respiratory gases, may be accounted for by the 3D surface reconstruction. The air velocity, temperature, humidity, and CO₂ concentration profiles of the air flowing into the test room can be measured to provide inflow boundary conditions for a computational fluid dynamics (CFD) simulation. Collected thermal imaging data may be included in the CFD analysis to provide basic geometrical models. Room airflow and ventilation may create turbulence around the exhaled CO₂ bubble. The air velocity, temperature, humidity, and CO₂ concentration profiles of the air flowing into the room may be measured to provide inflow boundary conditions for the CFD simulation.

A fast, background oriented Schlieren (BOS) camera system can be used to compute a 3D model of the surface of the exhaled respiratory gases. For example, a BOS imaging system by LaVision may include high-speed optical camera, lens, background material, one or more light sources. For example, LaVision provides digital image correlation (DIC) software and DaVis software for intelligent imaging, along with a BOS software package for computing the gas flow field and performing 3D reconstruction. Background features within the room can be used to align the images, and focal lengths may be adjusted.

The relevant information about the CO₂ bubble derived from the thermal imaging system can be fused onto the 3D model of exhaled respiratory gases derived from the optical BOS imaging system using a variety of well-known techniques. The resulting fused data representation integrates the CO₂ concentration with the exhaled gas volume in each breathing cycle, enabling parametric fitting to individual breathing patterns. The information extracted from the fusion of thermal information and 3D reconstructed volumes can be used to obtain the CO₂ concentration in exhaled respiratory gases, in addition to volume of exhaled respiratory gases. Furthermore, a flow analysis technique such as a 3D extension of optical flow can be used to analyze the fluid dynamics of exhaled respiratory gases and thus measure flow parameters of the exhaled respiratory gases.

The early warning system can provide an indication of dangerous levels of exhaled CO₂, and/or volume or rates of change using the data collected from the respiration imaging system. A mobile application may be implemented for real-time monitoring and abnormal condition notification. In some embodiments, a database server can receive information from the thermal imaging system and optical imaging system and store it in a database, allowing a user to view the image and related parameters at certain time points. The system can check whether the level of exhaled CO₂ is moving towards or reaching a dangerous level based on a threshold decided by the user or calibrated by previous data and show a warning message with detailed information.

Variables related to human subjects (e.g., movement, posture, temperature differences in exhaled respiratory gases, and sex differences in exhaled gas volumes) may also be accounted for by the application. Males and females have different breathing patterns and exhaled gas volumes. Data for a group of male and female participants can be collected in different postures (e.g., sitting, lying in bed, etc.) and used to optimize the algorithms to account for these human variables for accurate measurement of the respiratory volume, respiratory rate and CO₂ concentration of exhaled respiratory gases measured using the multi-sensor respiratory imaging system.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A system for monitoring an individual's respiration, comprising:
a thermal sensor configured to detect CO₂ gas;
at least one optical sensor; and
image processing circuitry configured to determine a parameter of CO₂ gas being exhaled about a mouth of the individual based upon a thermal image captured by the thermal sensor and at least one optical image captured by the at least one optical sensor.

2. The system of claim 1, further comprising processing circuitry configured to provide an indication of onset of acute respiratory distress syndrome (ARDS) in the individual based upon the parameter.

3. The system of claim 2, wherein the thermal sensor comprises a camera positioned about the individual, and the at least one optical sensor comprises at least one optical camera positioned about the individual.

4. The system of claim 3, wherein the thermal camera comprises a filter to detect the exhaled CO₂ gas.

5. The system of claim 4, wherein the filter is a bandpass filter with a center wavelength between about 4.1 μm and about 4.5 μm.

6. The system of claim 3, wherein the parameter is determined by a background-oriented schlieren (BOS) technique.

7. The system of claim 3, wherein the parameter is determined by combining information determined from the thermal image and information determined from the at least one optical image.

8. The system of claim 7, wherein the resolution of the at least one optical image is at least four times the resolution of the thermal image.

9. The system of claim 7, wherein the image processing circuitry determines a point cloud representation of a surface shape of the exhaled CO₂ gas.

10. The system of claim 7, further comprising early warning processing circuitry configured to provide an early warning indication in response to the exhaled CO₂ gas reaching or exceeding a predefined level of CO₂ concentration, CO₂ volume, or rate of change of the CO₂ concentration or CO₂ volume.

11. The system of claim 10, wherein the image processing circuitry and the early warning processing circuitry are remotely located.

12. The system of claim 10, wherein the early warning indication is communicated to a user device for display.

13. The system of claim 1, wherein the parameter comprises volume or three-dimensional (3D) information of the exhaled CO₂ gas.

14. The system of claim 1, wherein the parameter is based upon the thermal image and a plurality of optical images captured by the at least one optical sensor at a plurality of time points.

15. The system of claim 1, wherein the parameter is based upon a plurality of thermal images captured by the thermal sensor at a plurality of time points and the at least one optical image.

16. The system of claim 15, wherein the parameter comprises a rate of change of the exhaled CO₂ gas.

17. The system of claim 1, wherein the at least one optical sensor comprises a plurality of optical sensors, and the image processing circuitry is configured to determine the parameter of CO₂ gas being exhaled about a mouth of the individual based upon the thermal image captured by the thermal sensor and a plurality of optical images captured by the plurality of optical sensors.

18. The system of claim 17, wherein the parameter is determined by a background-oriented schlieren (BOS) technique.

19. The system of claim 18, wherein the parameter comprises volume or three-dimensional (3D) information of the exhaled CO₂ gas.

20. A system for monitoring an individual's respiration, comprising:
an arrangement of thermal and optical sensors configured to monitor respiration of an individual, where the thermal sensors are filtered to detect CO₂; and
image processing circuitry configured to determine a flow field or three-dimensional (3D) distribution of gas being exhaled about a mouth of the individual based upon thermal images and optical images captured by the arrangement of thermal and optical sensors.

21. The system of claim 20, comprising identifying onset of Acute Respiratory Distress Syndrome (ARDS) in the individual based at least in part upon the flow field or 3D distribution of gas.

22. The system of claim 21, wherein the arrangement of thermal and optical sensors comprises one or more thermal cameras and one or more optical cameras positioned about the individual.

23. The system of claim 22, wherein individual thermal cameras of the one or more thermal cameras comprise a filter to detect the CO₂.

24. The system of claim 23, wherein the filter is a bandpass filter.

25. The system of claim 22, wherein the optical images are provided by the one or more optical cameras with a background-oriented schlieren (BOS) technique.

26. The system of claim 20, wherein the flow field or 3D distribution of gas is determined by combining information determined from the thermal images and information determined from the optical images.

27. The system of claim 26, wherein the resolution of the optical images is at least four times the resolution of the thermal images.

28. The system of claim 26, wherein a point cloud representation of a surface shape of a bubble of the exhaled gas is generated based upon the optical images.

29. The system of 26, comprising processing circuitry configured to provide an early warning indication in response to the exhaled gas reaching or exceeding a predefined level of CO₂ concentration, CO₂ volume, or rate of change of the CO₂.

30. The system of claim 29, wherein a remotely located computing device comprises the processing circuitry configured to determine the flow field or 3D distribution of gas and provide the early warning indication.

31. The system of claim 29, wherein the early warning indication is communicated to a user device for display.

32. A system for monitoring an individual's respiration, comprising:
an arrangement of thermal sensors configured to monitor respiration of an individual wherein the thermal sensors are filtered to detect CO₂.

33. The system of claim 32, wherein the arrangement of thermal sensors comprises a plurality of thermal cameras positioned about the individual.

34. The system of claim 33, wherein individual thermal cameras of the plurality of thermal cameras comprise a filter to detect CO₂.

35. The system of claim 34, wherein the filter is a bandpass filter.

36. The system of claim 32, wherein a property of CO₂ gas being exhaled about the mouth of the individual is determined based upon thermal images captured by the arrangement of thermal sensors.

37. A system for monitoring an individual's respiration, comprising:
an arrangement of optical sensors configured to monitor gas flow being exhaled about a mouth of the individual.

38. The system of claim 37, wherein the arrangement of optical sensors comprises a plurality of optical cameras positioned about the individual.

39. The system of claim 38, wherein optical images are captured by the plurality of optical cameras with a background-oriented schlieren (BOS) technique.

40. The system of claim 39, wherein the gas flow being exhaled about the mouth of the individual is determined based upon the optical images.

41. The system of claim 40, wherein resolution of the optical images is at least one megapixel.

42. The system of claim 40, wherein a point cloud representation of a surface shape of a bubble of the gas flow being exhaled about the mouth of the individual is generated based upon the optical images.
